(19) United States
(12) Patent Application Publication
(10) Pub. No.: US 2010/0166891 A1
(43) Pub. Date: Jul. 1, 2010

(54) Composition comprising hemp oil for treating topical diseases, especially hand-foot syndrome

(75) Inventor: Timm Schmidt, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Intendis GmbH, Berlin (DE)

(21) Appl. No.: 12/640,795

(22) Filed: Dec. 17, 2009

Related U.S. Application Data
(60) Provisional application No. 61/140,132, filed on Dec. 23, 2008.

(30) Foreign Application Priority Data
Dec. 23, 2008 (EP) 08075967.3

(51) Int. Cl. A61K 36/00; A61P 17/00

(52) U.S. Cl. 424/725

(57) ABSTRACT

The invention relates to a novel composition comprising hemp oil for treating topical diseases, in particular the hand-foot syndrome.

This application claims the priority according to the Paris Convention of the European Patent application EP 08075967.3 (filing date: Dec. 23, 2008) as well as all benefits from earlier U.S. application Ser. No. 61/140,132 (filing date: Dec. 23, 2008), which are both incorporated herein by reference.

### PRIOR ART

When using cytostatics (chemotherapeutics such as for instance 5-fluorouracil (5-FU), gemcitabin, methotrexate, and others) in cancer therapy, often to the so-called hand-foot syndrome develops in patients. It is a palmar-plantar erythrodysesthesia. This skin lesion may easily affect the patient in a light to heavy manner in his daily life. The pathogenesis of the disease is unclear to a high degree. (Salzberg et al, Schweiz Med. Wochenschr. 2000; 130:1413-6). Treatment of the hand-foot syndrome is made for instance by administration of pain relieves, corticosteroids, D-panthenol or vitamin B6. In another attempt to treat the hand-foot syndrome, dosage adjustments of the respective cytostatic are proposed (Salzberg et al, see above). However, such dosis adjustment may affect the efficacy of the primary cancer treatment.

For the treatment of the hand-foot syndrome as a side effect during the treatment with capecitabine, further an ointment of hemp oil was proposed. This ointment was prepared on the basis of hemp oil with addition of different vegetable fats such as e.g. mango butter and shea butter or beeswax with further additions of D-panthenol, urea, bee honey, lavender oil and aloe vera gel (F. Kober, www.aekwien.at/media/BAEZ_{—}6Bez_modernetherapiekonzepte.pdf, no date, downloaded on Dec. 22, 2008). The ointment has however a low storage capability only (<3 months). The efficiency of the preparation decreases with time. Furthermore, this ointment has an unpleasant smell, which puts the patients off the regular application. Moreover, quite a number of cancer patients receiving chemotherapy suffer from smell disorders (“dysosmia”) (see e.g. Hong et al., J Support Oncol. 2009 March-April; 7(2):58-65, Ackerman et al., Pharmacotherapy 1997 May-June; 17(3):482-96, Epstein, Bone Marrow Transplant. 2002 December; 30(11):785-792). Probably as a result of such dysosmia a high number of patients stop treatment of the hand-foot syndrome by application of the above mentioned hemp oil ointment according to the prior art.

Further compositions comprising hemp oil are described in the following references:
WO 2004/108107, US 2008/286390, US 2008/233060, US 2007/280898,
US 2003/059489, U.S. Pat. No. 5,827,510, WO 95/31176.
However, none of these references describe the treatment of hand-foot syndrome. Moreover, none of the compositions disclosed in these references is able to overcome the problems relating to the olfactoric behavior of the compositions being unacceptable to quite a number of cancer patients.

For treating the hand-foot syndrome, there is therefore a need of novel preparations, which overcome the drawbacks of the prior art.

### GENERAL DESCRIPTION OF THE INVENTION

It has now been found that a preparation comprising hemp oil and at least one antioxidant overcomes the drawbacks of prior art and provides a composition, which is (among others) excellently suitable for treating the hand-foot syndrome.

The used hemp oil is a hemp oil obtained from hemp seeds (cannabis sativa seed oil). It contains no tetrahydrocannabinol (THC). It does however contain a series of unsaturated fatty acids, such as for instance linoleic acid, alpha-linoleic acid and gamma-linoleic acid. For the composition according to the invention it is decisive that the ratio linoleic acid+gamma-linoleic acid to alpha-linoleic acid is approximately 3:1. The share of the hemp oil in the composition according to the invention is approximately 10-40 weight percent, preferably 25-35 weight percent, particularly preferably 30 weight percent.

A special importance for the composition according to the invention is the presence of at least one antioxidant. It may for instance be selected from ascorbic acid, vitamin E (in particular tocotrienols), carotinoids (in particular lycopin), glutathione, butylhydroxytoluene and butylhydroxyanisole or a mixture of the mentioned substances. The preferred antioxidant is butylhydroxytoluene. The antioxidant will generally be comprised in a share of 0.01-0.5 weight percent, preferably 0.025-0.075 weight percent, particularly preferably approx. 0.05 weight to percent in the composition.

Optionally, still another preservation agent may be comprised. The preservation agent may be selected from the following group: phenoxyethanol, methylparaben, ethylparaben, propylparaben, benzyl alcohol or a mixture of the mentioned agents. Preferably, the preservation agent phenoxyethanol is added in a share of 0.2-2 weight percent, preferably 0.5-1.5 weight percent, particularly preferably approx. 1 weight percent.

The composition according to the invention is preferably prepared as a cream.

A particularly preferred embodiment of the composition according to the invention is a cream with the following composition:

| | |
|---|---|
| Cetearyl alcohol/cetearyl glucoside | approx. 7 g/100 g |
| Hemp oil | approx. 30 g/100 g |
| Phenoxyethanol | approx. 1 g/100 g |
| Butylhydroxytoluene | approx. 0.05 g/100 g |

which is adjusted by means of lactic acid to a pH value of approx. 5.5 and filled up with water to 100 g.

The composition according to the invention can be used for the topical treatment of human skin. The term “treatment” comprises the cosmetic as well as the therapeutic treatment of the human skin. Further, the prophylactic application is also comprised.

A special importance has the use of the composition according to the invention for treating the hand-foot syndrome. Affected patients feel after a short time already a relief of their afflictions, which will often completely disappear by application of the composition according to the invention.

By a prophylactic application of the composition according to the invention during the application of cytostatics, a significant reduction of the number of patients is observed that suffer from the hand-foot syndrome. Subject matter of the invention is therefore also a pharmaceutical kit comprising a chemotherapeutic and a composition according to at least one of claims 1-8. A kit is a unit, which contains a chemotherapeutic and a composition according to at least one of claims 1-8. The chemotherapeutic and the composition according to at least one of claims 1-8 may be separately packed, but are handed over commonly to the patient or the doctor or the clinic.

A preferred subject matter of the invention is a pharmaceutical kit, wherein the chemotherapeutic is selected from actinomycin D, actinomycin, alemtuzumab, alkylants, allicin, aminopterin, anthracycline, asparaginase, atrasentan, azacitidin, azathioprin, bevacizumab, bleomycin, bortezomib, bosutinib, busulfan, capecitabin, carboplatin, carmustin, cetuximab, chlorambucil, cilengitid, cisplatin, cladribin, colchicin, cyclophosphamid, cytarabin, dasatinib, daunorubicin, decitabine, docetaxel, doxifluridine, doxorubicin, epirubicin, epothilone, erlotinib, estramustine, etoposide, fludarabine, 5-fluorouracil, folic acid analogs, gemcitabine, gendicine, hydroxycarbamid, INNO-406, ibritumomab-tiuxetan, ifosfamide, imatinib, immucillin-H, irinotecan, lapatinib, lomustine, matuzumab, mechloroethamine, mercaptopurine, methotrexate, miltefosine, mitomycin, mitoxantrone, nilotinib, nimustine, oxaliplatin, paclitaxel, panitumumab, pemetrexed, podophyllotoxin, procarbazine, rituximab, sorafenib, stimuvax, streptozocin, temozolomid, teniposide, thiotepa, tioguanine, topotecan, trastuzumab, triptolide, vinblastine, vinca alkaloids, vincristine, vindesine, vinorelbine, zinostatin, in combination with a composition according to at least one of claims 1-9.

A particularly preferred subject matter of the invention is a pharmaceutical kit, wherein sorafenib in combination with a cream is comprised with the following composition:

| | |
|---|---|
| Cetearyl alcohol/cetearyl glucoside | approx. 7 g/100 g |
| Hemp oil | approx. 30 g/100 g |
| Phenoxyethanol | approx. 1 g/100 g |
| Butylhydroxytoluene | approx. 0.05 g/100 g |

which is adjusted by means of lactic acid to a pH value of approx. 5.5 and filled up with water to 100 g.

The object of the present invention is therefore a composition containing hemp oil, an antioxidans, a preservative together with pharmaceutically acceptable carriers and buffers bit without any further active drug compounds, such as antibiotics, anti-inflammatory agents or others. The composition according to the invention is therefore essentially free of any active drug compound.

The term “active drug compound” refers to compounds with proved pharmaceutical activity demonstrated in clinical trials and approved as a drug by the European Medicines Agency (EMEA) or the US Food and Drug Administration (FDA). The term “essentially free of active drug compound” means that no “active drug compound” has been intended to be added to the composition. The total amount of pharmaceutically active ingredients as a result of unintended contamination is therefore well below 0.05%, preferably below 0.01%. Most preferred is a composition in which no amount of any other active drug compound (besides the components of hemp oil and the antioxidans) can be detected with standard analytical methods used in pharmaceutical technology.

Surprisingly, it has further been found that the composition according to the invention is also suitable for treating other inflammatory skin diseases, such as psoriasis, atopic dermatitis, various kind of eczema and neurodermitis. Subject matter of the invention is therefore also the use of the composition according to the invention for treatment of these skin diseases. Best results are achieved in the treatment of the hand-foot syndrome, psoriasis, atopic dermatitis, especially in the hand-foot syndrome.

### Preparation

The cream is prepared by standard methods of the pharmaceutical technology. These are known to the expert skilled in the art and do not need any further explanation.

### EXAMPLES

### Example 1

The pharmaceutical composition in the form of a cream is mixed in a usual manner known to the man skilled in the art, in the following compositions (A-E). The pH value is respectively adjusted by means of lactic acid to 5.5.

| | Qty. | Qty. | Qty. | Qty. | Qty. |
|---|---|---|---|---|---|
| | [g/100 g] | [g/100 g] | [g/100 g] | [g/100 g] | [g/100 g] |
| Component | A | B | C | D | E |
| Cetearyl Alcohol/Cetearyl | 7 | 7 | 8 | 7 | 6 |
| Glucoside | | | | | |
| Hemp oil | 30 | 25 | 35 | 30 | 30 |
| Phenoxyethanol | 1 | 1 | 1 | 1 | 1 |
| Butylhydroxytoluene | 0.05 | 0.05 | 0.05 | 0.01 | 0.10 |
| Water | ad 100 | ad 100 | ad 100 | ad 100 | ad 100 |

As indicated above, the most preferred composition is the composition according to example 1A.

### Example 2

The compositions according to Example 1 A-E are stored over a longer time at an increased temperature. It is found that the composition according to the invention has a storage capability of clearly longer than 2 years.

### Example 3

Patients with the hand-foot syndrome are treated with the composition according to the invention. The application takes place several times per day, preferably at least three times per day at the affected locations. The patients show already after a 1-week application a distinct relief of the symptoms, in some patients the symptoms completely disappear after one week.

### Example 4

Cancer patients to be treated with sorafenib (INN, trade name Nexavar) obtain at the same time as the first administration of sorafenib the composition according to the invention. The application of the composition according to the invention takes place several times per day, preferably at least three times per day at the affected places. The patients show a significantly reduced tendency to the development of the hand-foot syndrome.

### Example 5

Patients with neurodermitis are treated several times per day with the composition according to the invention. After one week of application already a clear relief of the afflictions is detectable.

### Example 6a

A number of human volunteers is divided into three groups:
I) Healthy volunteers not suffering from any type of cancer
II) Patients recently diagnosed with various types of cancer, before receiving chemotherapy
III) Cancer patients (various types of cancer), receiving chemotherapy for at least 2 months

In a double-blind test these three groups of volunteers are presented either
a) a placebo cream (containing olive oil rather than hemp oil)
b) the hemp oil ointment according to the prior art (Kober, see section “prior art”, above) or
c) the hemp oil composition according to example 1A. Probands are asked to describe the smell of the composition as pleasant(1), neutral(2), unpleasant(3),unacceptable(4). The results are provided in FIG. 1: It is obvious that there in an unexpected preference of the composition according to the present invention over the prior art composition.

### Example 6b

A number of human probands is divided into three groups:
I) Healthy volunteers not suffering from any type of cancer
II) Patients recently diagnosed with various types of cancer, before receiving chemotherapy
III) Cancer patients (various types of cancer), receiving chemotherapy for at least 2 months

In a double-blind study these three groups of humans are offered either the hemp oil ointment according to the prior art (Kober, see section “prior art”, above) or the hemp oil composition according to example 1A for a one time application to their hands. In all three groups the vast majority of humans selects the composition according to example 1A. The percentage of probands selecting the composition according to example 1A is higher in group II and highest in group III.

### Example 7

In a placebo-controlled double-blind study cancer patients treated with chemotherapy for at least six weeks and suffering from hand-foot syndrome receive either the hemp oil ointment according to the prior art (Kober, see section “prior art”, above) or the hemp oil composition according to example 1A. Treatment is applied twice daily for six weeks. In the group receiving hemp oil ointment according to the prior art the majority of probands complain about the smell of their hands after treatment with the hemp oil treatment. Almost 50% of the probands stop treatment with the composition. In the group receiving the composition according to example 1A only a few probands describe an unpleasant smell, but none of the patients stops treatment. The majority of probands describe an improvement of the hand-foot syndrome, some patients even feel free of any symptoms thereof. In the placebo group none of the probands describe any unpleasant smell. However, also none of the patients describe any improvement of their hand-foot syndrome.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. application No. 61/140,132, filed Dec. 23, 2008, and EP application No. 08075967.3, filed Dec. 23, 2008, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### DESCRIPTION OF FIGURES

FIG. 1: Patients comparative olfactoric assessment of compositions Overall olfactoric assesment of compositions (a: placebo, b: prior art, c: composition according to example 1A), data provided as percentage of patients receiving a specific treatment

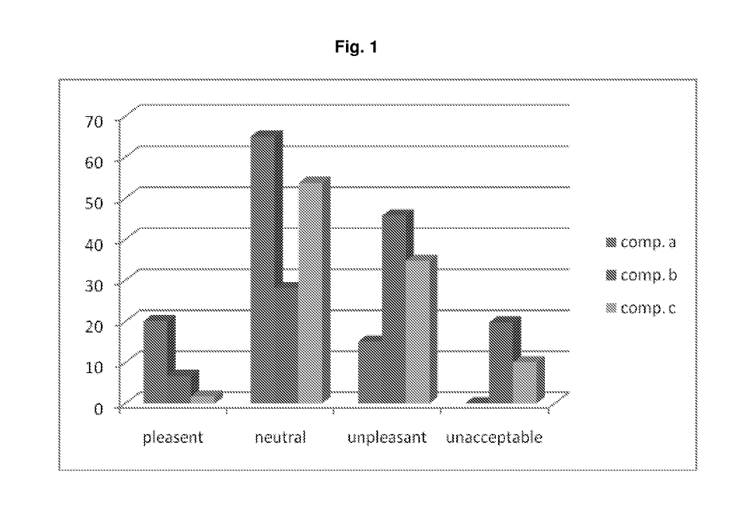

FIG. 2: Patients comparative olfactoric assessment of compositions Olfactoric assesment of compositions (a: placebo, b: prior art, c: composition according to example 1A), comparison of patent groups (I: healthy volunteers, II: untreated cancer patients, III: cancer patients treated with chemotherapy, data provided as percentage of patients within a specific patient group

FIG. 3: Patients comparative olfactoric assessment of compositions Olfactoric assesment of compositions (a: placebo, b: prior art, c: composition according to example 1A), comparison of patent groups (I: healthy volunteers, II: untreated cancer patients, III: cancer patients treated with chemotherapy, data provided as percentage of patients within a specific patient group

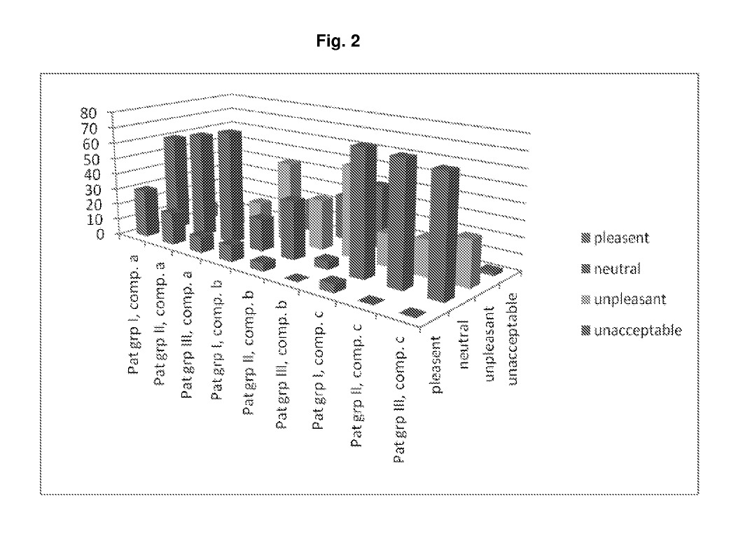

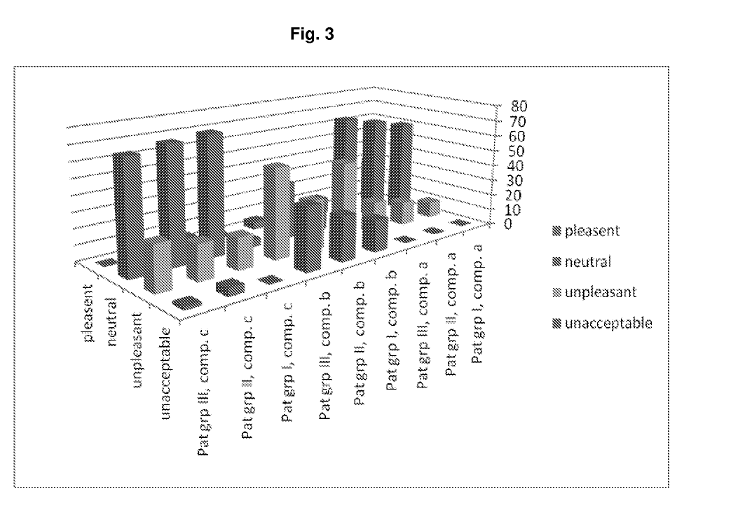

[same data as FIG. 2, different perspective]

1. A composition for the topical treatment of human skin in a dermatologically compatible carrier comprising
(a) hemp oil in an amount of 10-40 weight percent
(b) at least one antioxidant,
selected from ascorbic acid, vitamin E (in particular tocotrienols), carotinoids (in particular lycopin), glutathione, butylhydroxytoluene and butylhydroxyanisole or a mixture of the mentioned agents.

2. The composition according to claim 1 in the form of a cream.

3. The composition according to claim 1, wherein the hemp oil has a share of 25-35 weight percent.

4. The composition according to claim 1, wherein the hemp oil has a share of 30 weight percent.

5. The composition according to claim 1, wherein the antioxidant is butylhydroxytoluene, which has a share of 0.01-0.5 weight percent, preferably 0.025-0.075 weight percent, particularly preferably approx. 0.05 weight percent.

6. The composition according to claim 1, wherein in addition a preservation agent is contained, which is selected from phenoxyethanol, methylparaben, ethylparaben, propylparaben, benzyl alcohol or a mixture of the mentioned agents.

7. The composition according to claim 1, wherein the preservation agent is phenoxyethanol, which has a share of 0.2-2.0 weight percent, preferably 0.5-1.5 weight percent, particularly preferably approx. 1.0 weight percent.

8. The composition according to claim 1 in the form of a cream with the following composition:

| | |
|---|---|
| Cetearyl alcohol/cetearyl glucoside | approx. 7 g/100 g |
| Hemp oil | approx. 30 g/100 g |
| Phenoxyethanol | approx. 1 g/100 g |
| Butylhydroxytoluene | approx. 0.05 g/100 g |

which is adjusted by means of lactic acid to a pH value of approx. 5.5 and filled up with water to 100 g.

9. The use of a composition according to claim 1 for the manufacture of a medicament for the topical treatment of human skin.

10. The use according to claim 9 for treating the topical treatment of hand-foot syndrome, psoriasis, atopic dermatitis, various kinds of eczema and neurodermitis.

11. A pharmaceutical kit comprising a chemotherapeutic and a composition according to claim 1.

12. A pharmaceutical kit comprising sorafenib and a composition according to claim 8.

13. A method of treating a patient suffering from a skin disease by topical application of a composition according to claim 1.

14. A method of treating a patient suffering from a skin disease by topical application of a composition according to claim 1, wherein the skin disease is selected from hand-foot syndrome, psoriasis, atopic dermatitis, various kinds of eczema and neurodermitis by topical application of a composition according to claim 1.
